(19) United States
(12) Patent Application Publication
(10) Pub. No.: US 2026/0144493 A1
(43) Pub. Date: May 28, 2026

(54) OUTLIER CHANNEL IDENTIFICATION AND CORRECTION ON A BIN-BY-BIN BASIS FOR REAL-TIME ARTIFACT CORRECTION

(71) Applicant: Battelle Memorial Institute, Columbus, OH (US)

(72) Inventors: Nick TACCA, Columbus, OH (US); David A. Friedenberg, Worthington, OH (US); Collin DUNLAP, New Albany, OH (US); Bryan SCHLINK, Gahanna, OH (US)

(21) Appl. No.: 19/398,220

(22) Filed: Nov. 24, 2025

Related U.S. Application Data
(60) Provisional application No. 63/725,619, filed on Nov. 27, 2024.

(51) Int. Cl. A61B 5/00; G06F 18/2433; G06F 18/27

(52) U.S. Cl. CPC A61B 5/7214; A61B 5/7267; A61B 5/7278; G06F 18/2433; G06F 18/27

(57) ABSTRACT

In an approach to outlier channel identification and correction on a bin-by-bin basis for real time artifact correction, a system includes a computing device. The computing device is configured to: receive data; select a next bin of the data; for each bin of the data: identify one or more outlier channels; determine an artifact weighting of the one or more outlier channels; subtract the artifact weighting from one or more remaining features of the data; and replace the one or more outlier channels with interpolated data.

### CROSS-REFERENCE TO RELATED APPLICATIONS

The present application claims the benefit of the filing date of U.S. Provisional Application Ser. No. 63/725,619, filed Nov. 27, 2024, the entire teachings of which application is hereby incorporated herein by reference.

### STATEMENT REGARDING FEDERALLY SPONSORED RESEARCH OR DEVELOPMENT

This invention was made with government support under contract number W81XWH2211119 awarded by the Department of Defense-Congressionally Directed Medical Research Programs and the State of Ohio Third Frontier Funding. The government has certain rights in the invention.

### TECHNICAL FIELD

The present disclosure relates generally to signal processing and, more particularly, to a system and method for outlier channel identification and correction on a bin-by-bin basis for real-time artifact correction.

### BACKGROUND

Artifact reduction refers to the process of minimizing unwanted distortions or errors in data, such as electroencephalogram (EEG) signals, by employing algorithms like subtraction based on spatial EEG filtering methods like Principal Component Analysis (PCA) or Independent Component Analysis (ICA).

Regression analysis is a statistical method used for estimating the relationships between a dependent variable and independent variables. Regression analysis is a common tool in medical research data analysis and may be used in a variety of fields such as neurosurgical studies. Regression models can be used to construct clinical prediction rules that help to guide clinical decisions. Regression analysis is an important statistical method that is commonly used to determine the relationship between several factors and disease outcomes or to identify relevant prognostic factors for diseases. Regression analysis is an important statistical method for the analysis of medical data. It enables the identification and characterization of relationships among multiple factors. It also enables the identification of prognostically relevant risk factors and the calculation of risk scores for individual prognostication.

### BRIEF DESCRIPTION OF THE DRAWINGS

Reference should be made to the following detailed description which should be read in conjunction with the following figures, wherein like numerals represent like parts.

FIG. 1 is a functional block diagram illustrating a distributed data processing environment for outlier channel identification and correction on a bin-by-bin basis for real-time artifact correction consistent with the present disclosure.

FIGS. 2A and 2B are examples of artifact correction over a time-series segment of data consistent with the present disclosure.

FIGS. 3A and 3B are examples of a root-mean square (RMS) feature heatmap of an electromyogram functional electrical stimulation (EMG-FES) dataset without (FIG. 3A) and with (FIG. 3B) applying the channel correction filter, consistent with the present disclosure.

FIG. 4 is an example graph of a non-corrected signal versus various corrected signals consistent with the present disclosure.

FIGS. 5A and 5B are examples of real-time prediction probabilities from a spinal cord injury subject consistent with the present disclosure.

FIG. 6 is a flowchart diagram depicting operations for one illustrative embodiment of a workflow for outlier channel identification and correction on a bin-by-bin basis for real-time artifact correction, on the distributed data processing environment of FIG. 1, consistent with the present disclosure.

FIG. 7 depicts a block diagram of components of the computing device executing the workflow of FIG. 6 within the distributed data processing environment of FIG. 1, consistent with the present disclosure.

### DETAILED DESCRIPTION

Many current artifact filters require a separate training set to fit a model. A deep learning autoencoder approach, for example, uses training data to find a latent representation of the signal and then subsequently reconstruct the signal with the artifact suppressed. Similarly, blind source separation methods aim to decompose the original signal into spatially and/or spectrally uncorrelated sources. Outlier sources can be identified and suppressed in the reconstructed signal. With all of these approaches, however, a training set is required to find the outlier sources from the original signal so that they can be suppressed during inference.

Disclosed herein is a system and method for identifying and correcting outlier channels using regression analysis on a bin-by-bin basis for real-time artifact reduction. An advantage of the disclosed solution is that the outliers are determined on a real-time basis bin-by-bin (during inference). Therefore, if the signal tends to drift or unseen data that does not reflect the training data is used during inference, the disclosed channel correction filter can handle the new unseen data. Existing methods may not be able to adequately handle the new information and can potentially suppress important information from the signal. The alternative to artifact reduction is to leave the artifact in the signal and train models on the combined signal plus artifact data. However, research has shown that despite many machine learning methods being able to handle artifacts, if the artifact is removed prior to training a deep learning model, for example, the model is able to perform better than a model trained on a signal with an artifact. These findings were verified in an electromyogram functional electrical stimulation (EMG-FES) dataset in which decoding performance was improved by up to 5% bin-wise accuracy using the channel correction filter.

An EMG-FES system typically records electromyography and evokes functional movement with electrical stimulation. In some instances, the system records electromyography and evokes functional movement through the same electrodes. In other instances, the system typically records electromyography and evokes functional movement using separate EMG electrodes and FES patches on the same or nearby muscles. It should be noted that the disclosed system typically records electromyography and evokes functional movement through the same electrodes.

As used herein the term “signal” is a broad definition of the recorded data which will be processed by the disclosed system and method. A signal may include a single channel and/or sensor of data or multiple channels and/or sensors. In the description that follows, a group of channels and/or sensors may be described using the singular “signal” for clarity, since the group of channels and/or sensors in this context represents a single group of data.

Although the term “signal” is used in the examples that follow for clarity, the disclosed method is not restricted to signals recorded from multiple sensors per se. The disclosed method only requires that there are multiple channels and/or sensors and/or features. Although these terms are used interchangeably, this is just for ease of description.

When data is collected, it is collected in samples. The data may be collected from sensors (e.g., force sensors, pressure sensors, temperature sensors), channels (e.g., EMG, EEG channels), or by some other means to create features (e.g., stock prices, etc.). Therefore, in the illustrative example EMG case described herein with 70 EMG channels, the signal has a shape of n_samples×n_channels. As noted above, here the term “signal” refers to the whole body of data collected. In this example, samples of data are collected over time from the channels at a given sampling frequency.

In the disclosed method, instead of looking at each sample sequentially as it is being collected in time, a group of samples (termed a “bin” or a “window”) is analyzed to capture enough information to determine whether there are outliers in a bin. In this example, therefore, the EMG data may have the following shape: n_bins×bin_length×n_channels. Here, n_bins multiplied by bin_length=n_samples if the bin/windows are non-overlapping. They can be overlapping or non-overlapping depending on preference. Therefore, for example, if data is collected for 100 seconds, the disclosed method may assess bins in real-time that are 10 seconds long. In total, if using non-overlapping bins, there will be 10 bins that will have been assessed and/or corrected by the disclosed method. Overlapping bins may be used as well. Using the same example, if the bin length is kept as 10 seconds, but new bins are created every 5 seconds (rather than a full window=10 seconds), the bins will have a 50% overlap. In total, there will be 20 bins. For both cases, each bin of data will always have 70 EMG channels as before.

In this same example, it is the individual channel data and/or signals being corrected by the algorithm. The disclosed method is a way to assess within a window of multi-channel and/or sensor and/or feature data, which channels/sensors/features are outliers with respect to the rest of the channels and/or sensors and/or features. Then, using the clean and/or non-outlier channels and/or sensors and/or features, you can correct the outlier channels and/or sensors and/or features. Additionally, you can correct the clean and/or non-outlier channels and/or sensors and/or features by subtracting the artifact weighting from the outlier channels and/or sensors and/or features.

The disclosed channel correction filter analyzes a short segment or bin of data collected in real time. Each bin consists of a short time-series of feature data, for example, medical data such as EMG, EEG, neural data, categorical data, ECG, PPG, electronic health record information, etc. An artifact detection algorithm is used to identify outlier channels and/or features within the binned data. Non-limiting examples of artifact detection techniques include median absolute deviation (MAD), interquartile range, robust scale estimators, Gaussian mixture modeling, principal component analysis, k-nearest neighbors, etc. In the following examples, Median Absolute Deviation (MAD) was chosen as it is extremely fast and can be calculated in real-time without any prior training data used for fitting. This allows the filter to be run directly in inference-mode without requiring the filter to fit on a separate training dataset. Next, using regression analysis, the weighting of the artifact is subtracted from the remaining features. To remove the influence of the artifacts from the non-outlier features, the system first determines the weighting of artifact/outlier signal using Equation (1):

$${b}_{subtract} = {( {U}^{T} U )}^{- 1} {U}^{T} Y = {C}_{NN}^{- 1} {C}_{NY} \qquad ( 1 )$$

where U is the outlier/artifact signal, Y is the clean signal,

$${C}_{NN}^{- 1}$$

is the inverse of the auto-covariance matrix of the outlier/artifact signal, and C_{NY} is the cross-covariance between the artifact signal and clean signal. This assumes that the artifact is localized within the select features and that the artifact propagates throughout the rest of the feature data with lower amplitude. This may propagate the artifact throughout the mostly clean/unaffected features and hinder decoding performance. Therefore, subtracting the weighting of the artifact may help compensate for non-ideal features as a result of issues due to electrode conductance of the sensors, for example. The artifact/outlier signal is subtracted from the clean channels using Equation (2):

$${S}_{corrected} = Y - {Ub}_{subtract} \qquad ( 2 )$$

Next, the outlier channels are either removed from the signal or optionally (to keep the same size input), are interpolated based on neighboring channels or the full feature set. The artifact channels are interpolated with the neighboring channels using Equation (3):

$${b}_{keep} = {( {Y}^{T} Y )}^{- 1} {Y}^{T} {\overline{U}}_{N} \qquad ( 3 )$$

where Ū_{N} is the average neighboring features spatially surrounding artifact feature and Y is the clean signal. Then use weighting of neighboring channels to replace outlier features using Equation (4):

$${S}_{interpolated} = {Yb}_{keep} \qquad ( 4 )$$

A similar procedure is used with regression analysis of the neighboring/full feature weighting. However, instead of computing the cross-covariance between the clean signal and the outlier signal (as in the case when subtracting the artifact), the cross-covariance is computed from the clean signal and the average of the clean signal. This reduces the artifact completely from the time-series data. Subsequent features can be computed from the resulting filtered raw signal if desired.

Many people have approached artifact filters from different perspectives. Existing real time filters typically identify an artifact based on some threshold and correct an interpolation technique or remove the artifact altogether. The disclosed method of pairing artifact detection with regression analysis on a bin by bin basis has not been done before with time series data. This allows for retaining the same input data size after filtering, which is well suited to machine learning models that take a defined input number of features. Additionally, this allows for interpolating on the raw time series data rather than feature data. Many alternative approaches use averages of neighboring features, which is not suitable for raw biosignals (e.g., EMG or EEG) as the signals will cancel. Therefore, existing methods aside from regression analysis to interpolate the raw time-series information only in inference mode may not be suitable.

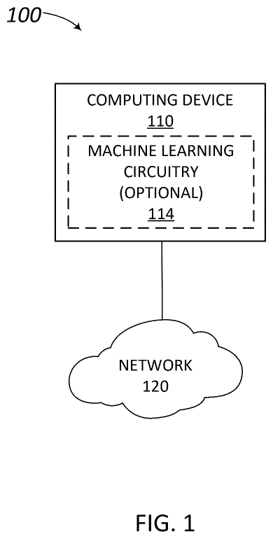

FIG. 1 is a functional block diagram illustrating a distributed data processing environment, generally designated 100, suitable for outlier channel identification and correction on a bin-by-bin basis for real-time artifact correction consistent with the present disclosure. The term “distributed” as used herein describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the disclosure as recited by the claims.

Distributed data processing environment 100 includes computing device 110 optionally connected to network 120. Network 120 can be, for example, a telecommunications network, a local area network (LAN), a wide area network (WAN), such as the Internet, or a combination of the three, and can include wired, wireless, or fiber optic connections. In general, network 120 can be any combination of connections and protocols that will support communications between computing device 110 and other computing devices (not shown) within distributed data processing environment 100.

In an embodiment, computing device 110 can be a standalone computing device, a management server, a web server, a mobile computing device, or any other electronic device or computing system capable of receiving, sending, and processing data. In another embodiment, computing device 110 can represent a server computing system utilizing multiple computers as a server system, such as in a cloud computing environment. In yet another embodiment, computing device 110 represents a computing system utilizing clustered computers and components (e.g., database server computers, application server computers) that act as a single pool of seamless resources when accessed within distributed data processing environment 100.

In an embodiment, the computing device 110 may include machine learning circuitry 114. In an embodiment, the machine learning circuitry 114 may be a neural network. In other embodiments, the machine learning circuitry 114 may be configured as a classifier, e.g., a tree-based classifier or a regression model. In an embodiment, the machine learning circuitry 114 may be the last step in the decoder pipeline which interprets the output (transformed signal) from all previous steps in the pipeline. The model is trained using standard ML practices. Training data is collected (signals and labels/targets, e.g. EMG signals and movement labels). The whole pipeline is fit on the training data and then it is either validated on a separate, held out dataset or used directly in the application (e.g., task practice for the EMG-FES system).

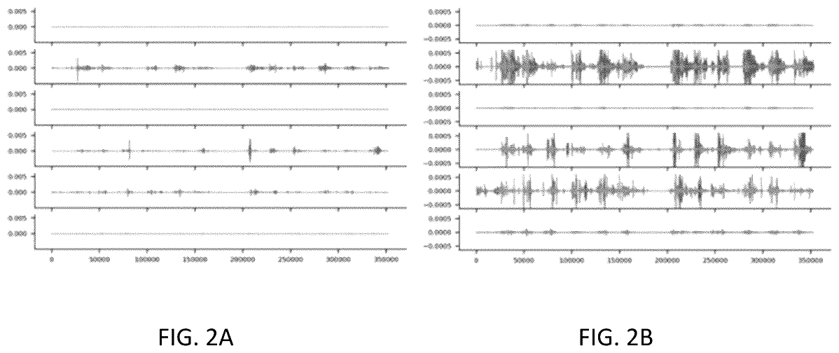

FIGS. 2A and 2B are an example of artifact correction over a time-series segment of data consistent with the present disclosure. For the example of FIGS. 2A and 2B, the data represents signals from an EMG. FIG. 2B represents the same signal from select channels as shown in FIG. 2A, but the signals in FIG. 2B are zoomed in to show greater detail.

FIGS. 2A and 2B illustrate an example of the artifact correction working over a long time series segment of data. The blue signal is the original EMG signal. The orange signal is the corrected EMG signal using the proposed channel correction filter, which corrects each bin of the data (in this particular case a 100 ms bin) in real-time.

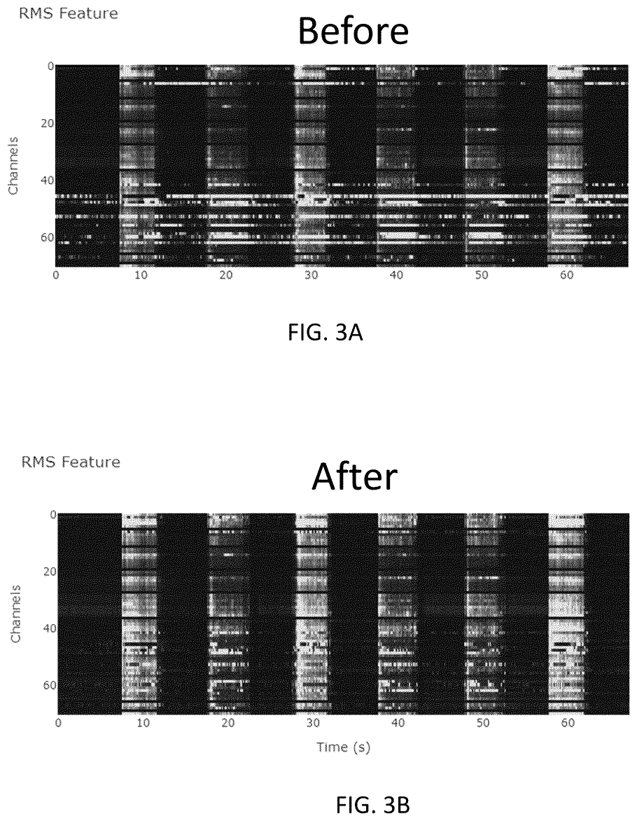

FIGS. 3A and 3B are examples of a root-mean square (RMS) feature heatmap of an EMG-FES signal before (FIG. 3A) and after (FIG. 3B) applying the channel correction filter. FIG. 3A shows the RMS calculated on the filtered EMG data without the disclosed channel correction filter. FIG. 3B shows the RMS calculated on the filtered EMG data with the channel correction filter. Artifact streaks, which are likely due to electrode lifting and/or a change in the conductance of the electrode, are noticeably removed from the heatmaps.

One illustrative example embodiment of the system and method for outlier channel identification and correction on a bin-by-bin basis for real time artifact correction is in EMG-FES. In this example, EMG signals are recorded using an EMG-FES sleeve. A decoder is then trained to interpret the EMG signals (in real-time) to predict motor intention. This predicted intention (or movement) is then actuated via functional electrical stimulation to evoke the desired motor intent of the user.

The disclosed channel correction filter is a single step within a greater decoder pipeline. The pipeline consists of individual steps that have an input and output and when chained together in a particular order produce a desired final output result (e.g., movement prediction). The channel correction step is crucial for the disclosed system in particular for two reasons. First, when stimulating with FES, there is a large artifact. The disclosed system uniquely stimulates and records through the same electrodes. In order to see the underlying signal to interpret intention while the FES is on, the artifact must be removed. Existing approaches involve subtracting a template artifact waveform or blanking out a particular part of the signal. However, in the existing approaches there were still residual artifacts (see FIG. 3A) due to changes in electrode contact, proximity to active FES electrodes, and the electrode conduction enhancer used. Second, the disclosed system incorporates various sleeve sizes and systems, across different individuals, with different electrode conduction enhancers (e.g., a proprietary MIEC sheet or hydrogel). It was found that with the proprietary MIEC sheet in particular, the signal became extremely noisy with these artifacts randomly appearing (even with existing artifact reduction techniques), due to small sheet movements/stretching, and changes in electrode contact. Unfortunately, this significantly reduced decoder accuracy (making the system difficult to use even with healthy individuals). The disclosed system addresses this issue using the final filtering step (i.e., the channel correction filter). This ensures all remaining artifacts are properly reduced to decode motor intent through the noisy signals. To summarize, it is the filtering step in the broader decoder pipeline that decodes motor intention (output) from EMG signals (input).

The disclosed channel correction filter can easily be extended to other applications. In some embodiments, the disclosed channel correction filter may be used with any device in which it is desired to control an FES continuously. So, for example, input signals from microelectrode arrays such as invasive Brain-Computer Interfaces (BCIs), EEG, magnetoencephalography (MEG), Electrocochleography (ECoG), functional near-infrared spectroscopy (fNIRS), Inertial Measurement Units (IMUs), etc. where the output is a decoded intention which triggers FES.

In some other embodiments, the disclosed channel correction filter may be used in any actuation, e.g., to control a prosthetic hand. When using EMG to control a grip for a prosthetic hand, an input is an EMG signal and an output is an intention used to trigger robotic hand. As the motors in the hand actuate, there may be artifacts due to changes in impedance of electrodes or electrical interference from the motors, etc., and the disclosed channel correction filter can be used to remove the artifacts.

In yet some other embodiments, the actuation piece is also not needed, and instead, those signals may be used to view/interpret muscle activity, compute biomarkers of motor function, track fatigue, etc. With neural recording systems, such as a microelectrode array, the disclosed channel correction filter may be used to understand neuron firing activity/networks, etc. While these cases would still involve biomarkers, in these cases the biomarker may be disease state or comprehension, etc., and the artifacts could arise by simply bumping into the electrodes or changes in impedance over time, etc.

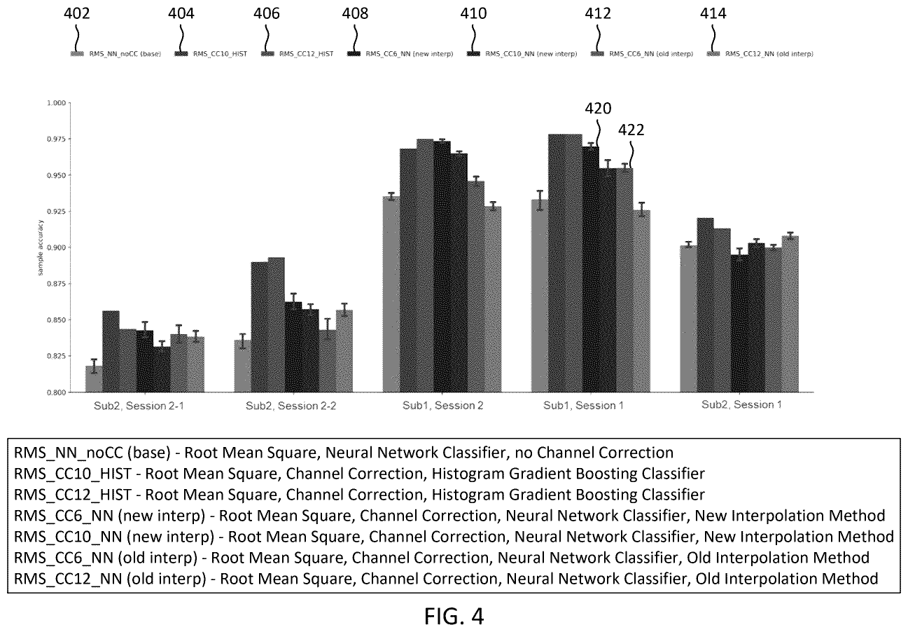

FIG. 4 is an example graph of a non-corrected signal versus various corrected signals consistent with the present disclosure. In the graph of FIG. 4, the vertical axis is the sample accuracy, where a value of one (1.000 in this example) means perfectly accurate. In the example graph of FIG. 4, the RMS_NN_noCC (base) 402 (i.e., Root Mean Square, Neural Network Classifier, no Channel Correction) is a base neural network (NN) model on non-corrected signal. All other bars use corrected signal with either a neural network classifier (RMS_CC6_NN (new interp) 408 and RMS_CC10_NN (new interp) 410, Root Mean Square, Channel Correction, Neural Network Classifier, New Interpolation Method) or histogram gradient boosting (HIST) classifier (RMS_CC10_HIST 404 and RMS_CC12_HIST 406, Root Mean Square, Channel Correction, Histogram Gradient Boosting Classifier). The green bars RMS_CC6_NN (old interp) 412 and RMS_CC12_NN (old interp) 414 (Root Mean Square, Channel Correction, Neural Network Classifier, Old Interpolation Method) use an older interpolation method that computed the cross-covariance between the artifact channels and clean channels. The updated method of RMS_CC6_NN (new interp) 408 and RMS_CC10_NN (new interp) 410 computes the cross-covariance between the average of neighboring channels and clean channels. This yields better performance, i.e., higher sample accuracy. For example, the result 420 shows a sample accuracy of almost 0.975 with the new interpolation method, while the result 422 with the same MAD threshold shows a sample accuracy of less than 0.950 with the old interpolation method. The numbers after channel correction (CC) denote different MAD thresholds. Adjusting the threshold allows you to control the sensitivity of the algorithm. Generally, a lower threshold indicates a better performance. Overall, the channel correction filter improves decoding performance within this EMG-FES dataset.

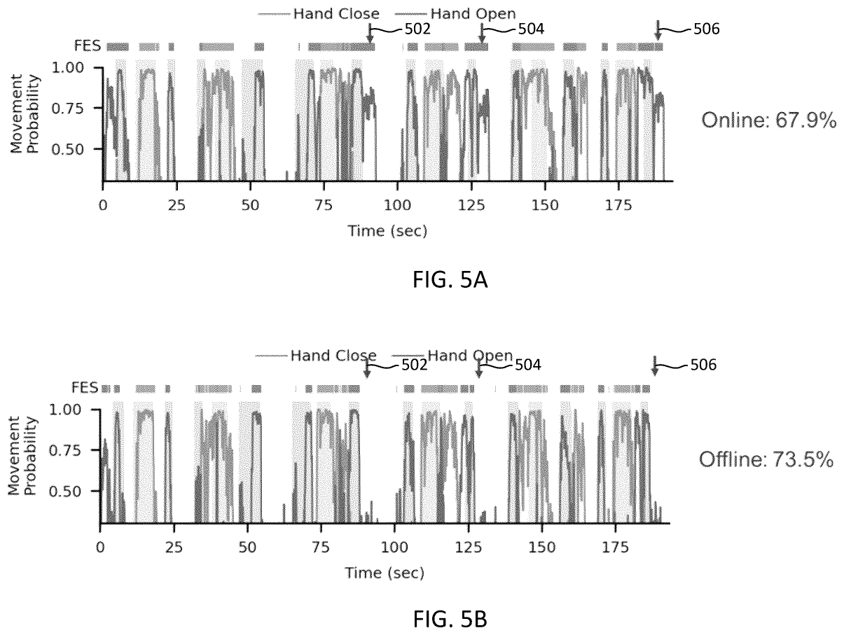

FIGS. 5A and 5B are examples of real-time prediction probabilities from a spinal cord injury subject consistent with the present disclosure. FIG. 5A shows a plot from the session without the disclosed channel correction filter applied. FIG. 5A includes cases at time-1 502, time-2 504, and time-3 506 in which the stimulation did not turn off even though the subject was queued to rest and was actively resting.

Applying the channel correction filter offline, the model predicts that those periods of prolonged stimulation when the subject was resting were actually resting periods. Therefore, when applied in real-time the stimulation would turn off. As shown in FIG. 5B, by removing artifacts, the decoders can learn to detect the “off” signal or “rest” signal better than when leaving artifacts. The rationale behind this stems from the decoder not knowing how to handle large inputs into the model. And thus, it assumes that burst of large artifact is volitional EMG and keeps the stimulation on, even though the subject is attempting to rest. FIG. 5B illustrates that at time-1 502, time-2 504, and time-3 506, with the disclosed channel correction filter applied, the stimulation did turn off.

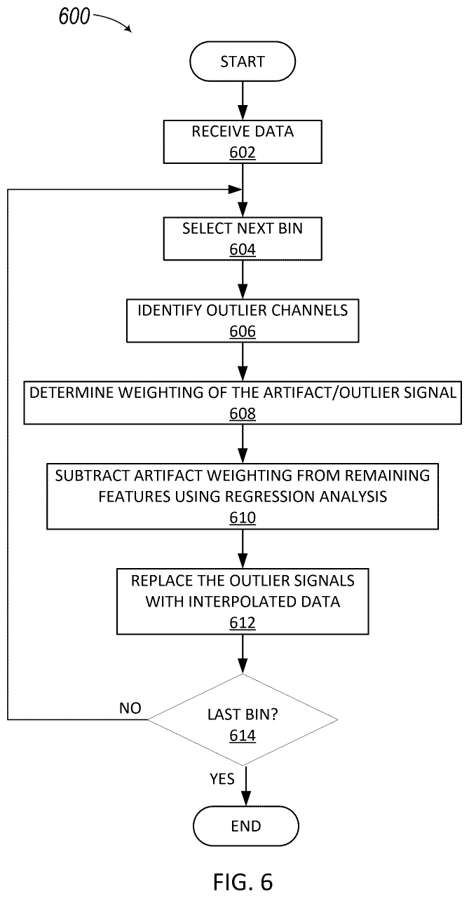

FIG. 6 is a flowchart diagram depicting operations for one illustrative embodiment of a workflow 600 for outlier channel identification and correction on a bin-by-bin basis for real-time artifact correction, on the distributed data processing environment of FIG. 1, consistent with the present disclosure. It should be appreciated that embodiments of the present disclosure provide at least for outlier channel identification and correction on a bin-by-bin basis for real-time artifact correction. However, FIG. 6 provides only an illustration of one implementation and does not imply any limitations with regard to the environments in which different embodiments may be implemented. Many modifications to the depicted environment may be made by those skilled in the art without departing from the scope of the disclosure as recited by the claims.

Receive data (operation 602). In the illustrated example embodiment, the process receives data that is collected and requires filtering to remove outlier channels for real-time artifact correction. The disclosed method works for any time-series data, which may include real-time data or data collected offline. The time-series data may be signals (e.g., biosignals which may include, but are not limited to, EMG, EEG, PPG, ECG, etc. or any other signals which may include, but are not limited to, kinematic, temperature, pressure, force, etc.). In some embodiments, the data may be categorical (e.g., medical health information received over time).

Select the next bin (operation 604). The received data is segmented into short bins of data, i.e., a short time-series of feature data, for example, medical data such as EMG, EEG, neural data, categorical data, or any other data requiring filtering. In an embodiment, the size of the bin may be predetermined. In another embodiment, the size of the bins may be user selectable.

Identify the outlier channels (operation 606). An artifact detection algorithm is used to identify outlier channels/features within the binned data. In an embodiment, the artifact detection algorithm may be MAD.

Determine weighting of the artifact/outlier signal (operation 608). To remove the influence of the artifacts from the non-outlier features, the system determines the weighting of the artifact/outlier signal. In an embodiment, the system may determine the weighting of the artifact/outlier signal using Equation (1) above.

Subtract artifact weighting from the remaining features using regression analysis (operation 610). Next, using regression analysis, the weighting of the artifact is subtracted from the remaining features to help compensate for non-ideal features as a result of issues due to factors such as electrode conductance of the sensors, for example. In an embodiment, the system may subtract the weighting of the artifact from the remaining features using Equation (2) above.

Replace the outlier signals with interpolated data (operation 612). The disclosed method performs the interpolation without including any artifact signal, which leads to better performance. Instead, the artifact channels are replaced with an interpolated signal of surrounding “clean” channels or the full clean signal using regression analysis. In an embodiment, regression analysis may be used for this technique to get the weighting of the clean signal and an average of the clean signal. As used herein, clean signal refers to one or more neighboring channels that are deemed “non-artifact” or the remaining “clean” (non-artifact) signal.

In an embodiment, a similar procedure may be used with regression analysis of the neighboring/full feature weighting. However, instead of computing the cross-covariance between the clean signal and the outlier signal (as in the case when subtracting the artifact), the cross-covariance is computed from the clean signal and the average of the clean signal. In an embodiment, the cross-covariance is computed from the clean signal and the average of the clean signal using Equation (3). This weighting is then used to interpolate the clean signals from the entire dataset or neighboring channels to replace the artifact channels for that bin. This reduces the artifact completely from the time-series data. Subsequent features can be computed from the resulting filtered raw signal.

Determine if the last bin was processed (decision block 614). If it is determined that the last bin was not processed (“no” branch, decision block 614), then the process returns to operation 604 to select the next bin to be processed. If it is determined that the last bin was processed (“yes” branch, decision block 614), then the process ends for this cycle.

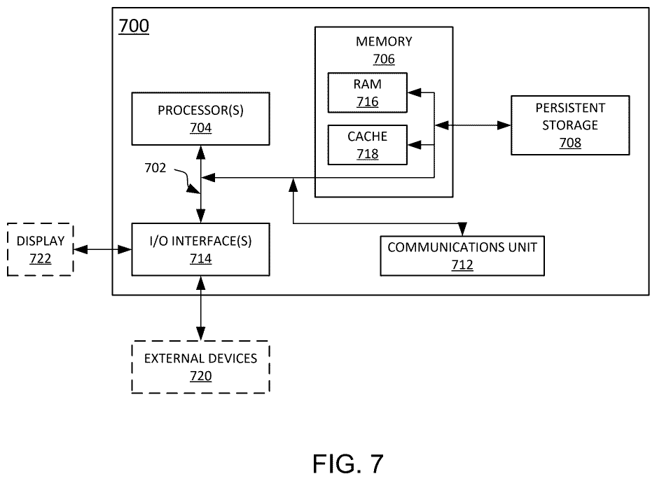

FIG. 7 is a block diagram depicting components of one example of a computing device 110 suitable for executing the workflow of FIG. 6 for outlier channel identification and correction on a bin-by-bin basis for real-time artifact correction within the distributed data processing environment of FIG. 1, consistent with the present disclosure. FIG. 7 displays the computing device or computer 700, one or more processor(s) 704 (including one or more controllers or computer processors), a communications fabric 702, a memory 706 including, a random-access memory (RAM) 716 and a cache 718, a persistent storage 708, a communications unit 712, I/O interfaces 714, a display 722, and external devices 720. It should be appreciated that FIG. 7 provides only an illustration of one embodiment and does not imply any limitations with regard to the environments in which different embodiments may be implemented. Many modifications to the depicted environment may be made.

As depicted, the computer 700 operates over the communications fabric 702, which provides communications between the computer processor(s) 704, memory 706, persistent storage 708, communications unit 712, and input/output (I/O) interface(s) 714. The communications fabric 702 may be implemented with an architecture suitable for passing data or control information between the processors 704 (e.g., microprocessors, communications processors, and network processors), the memory 706, the external devices 720, and any other hardware components within a system. For example, the communications fabric 702 may be implemented with one or more buses.

The memory 706 and persistent storage 708 are computer readable storage media. In the depicted embodiment, the memory 706 comprises a RAM 716 and a cache 718. In general, the memory 706 can include any suitable volatile or non-volatile computer readable storage media. Cache 718 is a fast memory that enhances the performance of processor(s) 704 by holding recently accessed data, and near recently accessed data, from RAM 716.

Program instructions for outlier channel identification and correction on a bin-by-bin basis for real-time artifact correction may be stored in the persistent storage 708, or more generally, any non-transitory computer readable storage media, for execution by one or more of the respective computer processors 704 via one or more memories of the memory 706. The persistent storage 708 may be a magnetic hard disk drive, a solid-state disk drive, a semiconductor storage device, flash memory, read only memory (ROM), electronically erasable programmable read-only memory (EEPROM), or any other computer readable storage media that is capable of storing program instruction or digital information.

The media used by persistent storage 708 may also be removable. For example, a removable hard drive may be used for persistent storage 708. Other examples include optical and magnetic disks, thumb drives, and smart cards that are inserted into a drive for transfer onto another computer readable storage medium that is also part of persistent storage 708.

The communications unit 712, in these examples, provides for communications with other data processing systems or devices. In these examples, the communications unit 712 includes one or more network interface cards. The communications unit 712 may provide communications through the use of either or both physical and wireless communications links. In the context of some embodiments of the present disclosure, the source of the various input data may be physically remote to the computer 700 such that the input data may be received, and the output similarly transmitted via the communications unit 712.

The I/O interface(s) 714 allows for input and output of data with other devices that may be connected to computer 700. For example, the I/O interface(s) 714 may provide a connection to external device(s) 720 such as a keyboard, a keypad, a touch screen, a microphone, a digital camera, and/or some other suitable input device. External device(s) 720 can also include portable computer readable storage media such as, for example, thumb drives, portable optical or magnetic disks, and memory cards. Software and data used to practice embodiments of the present disclosure can be stored on such portable computer readable storage media and can be loaded onto persistent storage 708 via the I/O interface(s) 714.

I/O interface(s) 714 may also connect to a display 722. Display 722 provides a mechanism to display data to a user and may be, for example, a computer monitor. Display 722 can also function as a touchscreen, such as a display of a tablet computer.

According to one aspect of the disclosure there is thus provided a system for outlier channel identification and correction on a bin-by-bin basis for real time artifact correction. The system includes a computing device. The computing device is configured to: receive data; select a next bin of the data; for each bin of the data: identify one or more outlier channels; determine an artifact weighting of the one or more outlier channels; subtract the artifact weighting from one or more remaining features of the data; and replace the one or more outlier channels with interpolated data.

According to another aspect of the disclosure, there is thus provided a method of outlier channel identification and correction on a bin-by-bin basis for real time artifact correction. The method includes receiving data; selecting a next bin of the data; for each bin of the data: identifying one or more outlier channels; subtracting artifact weighting from one or more remaining features of the data; and replacing the one or more outlier channels with interpolated data.

Although the methods and systems have been described relative to a specific embodiment thereof, they are not so limited. Obviously, many modifications and variations may become apparent in light of the above teachings. Many additional changes in the details, materials, and arrangement of parts, herein described and illustrated, may be made by those skilled in the art. Also, it may be appreciated that the phraseology and terminology used herein is for the purpose of description and should not be regarded as limiting as such may be understood by one of skill in the art. Throughout the present disclosure, like reference characters may indicate like structure throughout the several views, and such structure need not be separately discussed. Furthermore, any particular feature(s) of a particular exemplary embodiment may be equally applied to any other exemplary embodiment(s) of this disclosure as suitable. In other words, features between the various exemplary embodiments described herein are interchangeable, and not exclusive.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

“Circuitry,” as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry and/or future computing circuitry including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DSP), field programmable gate array (FPGA), logic gates, registers, semiconductor device, chips, microchips, chip sets, etc.

The term “coupled” as used herein refers to any connection, coupling, link, or the like by which signals carried by one system element are imparted to the “coupled” element. Such “coupled” devices, or signals and devices, are not necessarily directly connected to one another and may be separated by intermediate components or devices that may manipulate or modify such signals.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles “a” and/or “an” and/or “the” to modify a noun may be understood to be used for convenience and to include one, or more than one, of the modified noun, unless otherwise specifically stated. The terms “comprising”, “including” and “having” are intended to be inclusive and mean that there may be additional elements other than the listed elements.

The programs described herein are identified based upon the application for which they are implemented in a specific embodiment of the disclosure. However, it should be appreciated that any particular program nomenclature herein is used merely for convenience, and thus the disclosure should not be limited to use solely in any specific application identified and/or implied by such nomenclature.

The present disclosure may be a system, a method, and/or a computer program product. The system or computer program product may include one or more non-transitory computer readable storage media having computer readable program instructions thereon for causing a processor to carry out aspects of the present disclosure.

The one or more non-transitory computer readable storage media can be any tangible device that can retain and store instructions for use by an instruction execution device. The one or more non-transitory computer readable storage media may be, for example, but is not limited to, an electronic storage device, a magnetic storage device, an optical storage device, an electromagnetic storage device, a semiconductor storage device, or any suitable combination of the foregoing. A non-transitory computer readable storage media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from one or more non-transitory computer readable storage media or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in one or more non-transitory computer readable storage media within the respective computing/processing device.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some embodiments, electronic circuitry including, for example, programmable logic circuitry, Field-Programmable Gate Arrays (FPGA), or other Programmable Logic Devices (PLD) may execute the computer readable program instructions by utilizing state information of the computer readable program instructions to personalize the electronic circuitry, in order to perform aspects of the present disclosure.

It will be appreciated by those skilled in the art that any block diagrams herein represent conceptual views of illustrative circuitry embodying the principles of the disclosure. Similarly, it will be appreciated that any block diagrams, flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A system for outlier channel identification and correction on a bin-by-bin basis for real-time artifact correction, the system comprising:
a computing device, the computing device configured to:
receive data;
select a next bin of the data;
for each bin of the data:
identify one or more outlier channels;
determine an artifact weighting of the one or more outlier channels;
subtract the artifact weighting from one or more remaining features of the data; and
replace the one or more outlier channels with interpolated data.

2. The system of claim 1, wherein the system is configured to correct artifacts in an electromyogram functional electrical stimulation (EMG-FES) system.

3. The system of claim 2, further comprising a machine learning circuitry.

4. The system of claim 3, wherein the machine learning circuitry is at least one of a classifier, a tree-based classifier, or a regression model.

5. The system of claim 3, wherein the machine learning circuitry is trained on EMG signals and movement labels.

6. The system of claim 1, wherein identify the one or more outlier channels further comprises:
use an artifact detection technique chosen from a group consisting of a median absolute deviation (MAD), an interquartile range, a robust scale estimator, a Gaussian mixture modeling, a principal component analysis, and a k-nearest neighbors to identify the one or more outlier channels within each bin of the data.

7. The system of claim 1, wherein subtract the artifact weighting from the one or more remaining features of the data further comprises:
use regression analysis to subtract the artifact weighting from the one or more remaining features of the data to compensate for one or more non-ideal features of the data.

8. The system of claim 1, wherein the one or more outlier channels are replaced based on a regression analysis using weights to interpolate a neighboring clean signal.

9. The system of claim 1, wherein the one or more outlier channels are replaced based on a regression analysis using weights to interpolate a full clean signal.

10. The system of claim 1, wherein the one or more outlier channels are replaced based on a regression analysis using weights to interpolate a one or more remaining clean signals.

11. A method for outlier channel identification and correction on a bin-by-bin basis for real-time artifact correction, the method comprising:
receiving data;
selecting a next bin of the data;
for each bin of the data:
identifying one or more outlier channels;
subtracting artifact weighting from one or more remaining features of the data; and
replacing the one or more outlier channels with interpolated data.

12. The method of claim 11, wherein identifying the one or more outlier channels further comprises:
using an artifact detection technique chosen from a group consisting of a median absolute deviation (MAD), an interquartile range, a robust scale estimator, a Gaussian mixture modeling, a principal component analysis, and a k-nearest neighbors to identify the one or more outlier channels within each bin of the data.

13. The method of claim 11, wherein subtracting the artifact weighting from the one or more remaining features of the data further comprises:
using regression analysis to subtract the artifact weighting from the one or more remaining features of the data to compensate for one or more non-ideal features of the data.

14. The method of claim 11, wherein the one or more outlier channels are replaced based on a regression analysis using weights to interpolate a neighboring clean signal.

15. The method of claim 11, wherein the one or more outlier channels are replaced based on a regression analysis using weights to interpolate a full clean signal.

16. The method of claim 11, wherein the one or more outlier channels are replaced based on a regression analysis using weights to interpolate a one or more remaining clean signals.

17. The method of claim 11, wherein one or more artifacts are corrected in an electromyogram functional electrical stimulation (EMG-FES) system.

18. The method of claim 17, wherein subtracting the artifact weighting from the one or more remaining features of the data uses machine learning circuitry.

19. The method of claim 18, wherein the machine learning circuitry is at least one of a classifier, a tree-based classifier, or a regression model.

20. The method of claim 18, wherein the machine learning circuitry is trained on EMG signals and movement labels.
